(19) United States
(12) Patent Application Publication
(10) Pub. No.: US 2024/0252084 A1
(43) Pub. Date: Aug. 1, 2024

(54) STRESS RELEASE ACTION DETECTION DEVICE, STRESS RELEASE ACTION DETECTION METHOD, AND STORAGE MEDIUM

(71) Applicant: NEC Corporation, Minato-ku, Tokyo (JP)

(72) Inventors: Terumi UMEMATSU, Tokyo (JP); Keisuke SUZUKI, Tokyo (JP); Masanori TSUJIKAWA, Tokyo (JP)

(73) Assignee: NEC Corporation, Minato-ku, Tokyo (JP)

(21) Appl. No.: 18/567,136

(22) PCT Filed: Jun. 10, 2021
(86) PCT No.: PCT/JP2021/022119

§ 371 Date: Dec. 5, 2023

(51) Int. Cl. A61B 5/16; A61B 5/00; A61B 5/11; G16H 20/30

(52) U.S. Cl. CPC A61B 5/165; A61B 5/1123; A61B 5/1126; A61B 5/7405; A61B 5/742; G16H 20/30; A61B 2562/0219

(57) ABSTRACT

An information processing device 1X mainly includes a stress value acquisition means 17X, a stress release action detection means 18X, and a notification means 19X. The stress value acquisition means 17X acquires a stress value representing a degree of stress of a target person. The stress release action detection means 18X detects a stress release action, which is an action for releasing a stress, based on the stress value. The notification means 19X makes a notification of a detection result of the stress release action.

### TECHNICAL FIELD

The present disclosure relates to the technical field of an information processing device, a control method, and a storage medium for performing processing related to stress states.

### BACKGROUND

There is known a device or system for determining a stress state of a subject based on data measured from the subject. For example, Patent Literature 1 discloses a portable stress measuring device which determines a temporary stress degree of a subject on every day based on examination data regarding the subject. Further, Patent Literature 2 discloses a technique for calculating the amount of exercise from acceleration information obtained by an acceleration sensor.

### CITATION LIST

### Patent Literature

Patent Literature 1: JP2007-275287A
Patent Literature 2: JP2019-030389A

### SUMMARY

### Problem to be Solved

To maintain and improve mental health, it is essential to avoid getting stressed out and cope with the stress (i.e., release the stress) when the stress is built up. On the other hand, according to Patent Literature 1, the current stress state is merely determined and reported, and therefore it is difficult for the user to clearly grasp, from the notification regarding the current stress state, whether or not the stress has been relieved.

In view of the above-described issue, it is therefore an example object of the present disclosure to provide an information processing device, a stress estimation method, and a storage medium capable of suitably making a notification of information on a stress state regarding a subject.

### Means for Solving the Problem

In one mode of the information processing device, there is provided an information processing device including:
a stress value acquisition means configured to acquire a stress value representing a degree of stress of a target person;
a stress release action detection means configured to detect a stress release action, which is an action for releasing a stress, based on the stress value; and
a notification means configured to make a notification of a detection result of the stress release action.

In one mode of the control device, there is provided a control method executed by a computer, the control method including:
acquiring a stress value representing a degree of stress of a target person;
detecting a stress release action, which is an action for releasing a stress, based on the stress value; and
making a notification of a detection result of the stress release action. It is noted that the “computer” includes any electronic device (may be a processor included in the electronic device) and may be configured by a plurality of electronic devices.

In one mode of the storage medium, there is provided a storage medium storing a program executed by a computer, the program causing the computer to
acquire a stress value representing a degree of stress of a target person;
detect a stress release action, which is an action for releasing a stress, based on the stress value; and
make a notification of a detection result of the stress release action.

### Effect

An example advantage according to the present invention is to suitably make a notification of information on a stress state regarding a subject.

### BRIEF DESCRIPTION OF THE DRAWINGS

FIG. 1 It shows a schematic configuration of a stress release detection system according to a first example embodiment.

FIG. 2 It shows an example of a hardware configuration of the information processing device common to each example embodiment.

FIG. 3 It is an example of a functional block diagram of the information processing device according to the first example embodiment.

FIG. 4 It shows a two-dimensional map with sympathetic activity on the vertical axis and parasympathetic activity on the horizontal axis.

FIG. 5 It shows a two-dimensional map with the stress value on the vertical axis and the amount of exercise on the horizontal axis.

FIG. 6 It is a diagram schematically illustrating the stress release index, the lower limit threshold value, and the upper limit threshold value.

FIG. 7 It is an example of a stress release confirmation screen image.

FIG. 8 It is an example of a flowchart that the information processing device executes in the first example embodiment.

FIG. 9 It shows a schematic configuration of a stress release detection system in the second example embodiment.

FIG. 10 It is a block diagram of an information processing device according to a third example embodiment.

FIG. 11 It shows an example of a flowchart executed by the information processing device according to the third example embodiment.

### EXAMPLE EMBODIMENTS

Hereinafter, example embodiments of an information processing device, a stress estimation method, and a storage medium will be described with reference to the drawings.

### First Example Embodiment

### (1) System Configuration

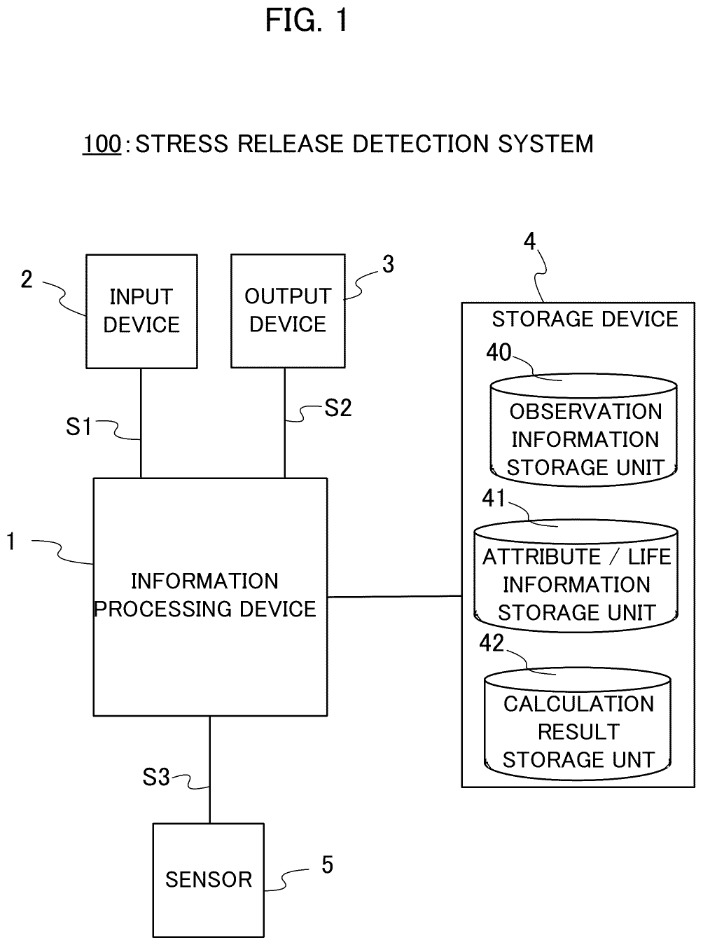

FIG. 1 shows a schematic configuration of a stress release detection system 100 according to a first example embodiment. The stress release detection system 100 detects an action (also referred to as “stress release action”) of a target person that promotes release of the stress of the target person, and makes a notification regarding the detection result. Here, the “target person” may be a sports player or an employee whose stress state is managed by an organization, or may be an individual user. The above-described “organization” may be a family. In this case, the stress release detection system 100 detects a stress release action for each member of the family as a target person and makes a notification of the detection result.

The stress release detection system 100 mainly includes an information processing device 1, an input device 2, an output device 3, a storage device 4, and a sensor 5.

The information processing device 1 detects a stress release action of the target person and notifies a user that is the target person or the manager of the detection result. The information processing device 1 performs data communication with the input device 2, the output device 3, and the sensor 5 through a communication network or through wireless or wired direct communication. For example, the information processing device 1 receives: an input signal “S1” supplied from the input device 2; a sensor signal “S3” supplied from the sensor 5; and various information stored in the storage device 4. The input signal S1 and the sensor signal S3 are used to generate information (also referred to as “observation information”) obtained by observing (measuring) the target person subjectively or objectively.

In the present example embodiment, the information processing device 1 estimates the stress state (specifically, the stress value representing the degree of stress) of the target person and calculates the amount of exercise of the target person based on the observation information, and detects the stress release action based on the calculation result. The information processing device 1 generates the output control signal “S2” based on the detection result of the stress release action of the target person and supplies the generated output control signal S2 to the output device 3. In the present example embodiment, the stress refers to a short-term stress that is, for example, a stress in a relatively short period (several seconds to several days).

The input device 2 is an interface that receives a user input (manual input) of information regarding each target person. The user who performs input of information using the input device 2 may be the target person itself, or may be a person who manages the activity of the target person. The input device 2 may be a variety of user input interfaces such as, for example, a touch panel, a button, a keyboard, a mouse, and a voice input device. The input device 2 supplies the input signal S1 generated based on the input from the user to the information processing device 1. The output device 3 displays and/or output by audio information based on the output control signal S2 supplied from the information processing device 1. The output device 3 includes, for example, a display device, such as a display and a virtual (augmented) real terminal and a projector, and an audio output device such as a speaker.

The sensor 5 measures a biological signal or the like of the target person and supplies the measured biological signal or the like to the information processing device 1 as a sensor signal S3. In this instance, the sensor signal S3 may be any biological signal (including vital information) regarding the target person such as a heart rate, EEG, pulse wave, sweating volume (skin electrical activity), amount of hormonal secretion, cerebral blood flow, blood pressure, body temperature, myoelectric potential, respiration rate, and acceleration. The sensor 5 may also be a device that analyzes blood collected from the target person and outputs a sensor signal S3 indicative of the analysis result. Examples of the sensor 5 include a wearable terminal worn by the target person, a camera for photographing the target person, a microphone for generating a voice signal of the target person's utterance, and a terminal such as a personal computer or a smartphone operated by the target person. For example, the above-described wearable terminal includes a GNSS (global navigation satellite system) receiver, an acceleration sensor, a sensor for detecting biological signals, and the like, and outputs the output signals from each sensor as a sensor signal S3. The sensor 5 may supply information corresponding to the manipulation amount signal from a personal computer or a smartphone to the information processing device 1 as the sensor signal S3. The sensor 5 may also output a sensor signal S3 representing biomedical data (including sleep time) regarding the target person during the sleep.

The storage device 4 is a memory for storing various information necessary for estimating the stress state. The storage device 4 may be an external storage device, such as a hard disk, connected to or embedded in the information processing device 1, or may be a storage medium, such as a flash memory. The storage device 4 may be a server device that performs data communication with the information processing device 1. Further, the storage device 4 may be configured by a plurality of devices.

The storage device 4 includes an observation information storage unit 40, an attribute/life information storage unit 41, and a calculation result storage unit 42.

The observation information storage unit 40 stores observation information that is subjective information regarding the target person based on the input signal S1 or objective information regarding the target person based on the sensor signal S3. Here, the sensor signal S3 itself may be used as observation information, or the features thereof calculated based on the sensor signal S3 (including an index representing the facial expression, emotion, and the like analyzed from an image or audio data) may be used as observation information. Further, the observation information may include questionnaire answer information based on the input signal S1 or a diagnostic result regarding a personality or the like based on the questionnaire answer information. For example, the observation information is stored in the observation information storage unit 40 in association with the identification information (subject ID) of the target target person to be observed and the observation date and time information.

The attribute/life information storage unit 41 stores at least one of attribute information regarding the attribute of the target person and/or life information regarding the life (lifestyle) of the target person.

Examples of the attribution information include preference information regarding whether the target person likes or dislikes exercises and information relating to gender, age, personality, race, or tendency of cognition regarding the target person. The attribute information may be generated by the information processing device 1 and stored in the storage device 4, or may be generated in advance by a device other than the information processing device 1 and stored in the storage device 4. For example, the attribute information is generated based on the answer result (i.e., subjective measurement result) of the questionnaire by the target person.

Examples of the life information includes: information on daily activity, which is the average amount of exercise of the target person on a daily basis; information on the target person's schedule (work days, working hours, travel days, etc.); information on his/her physical condition (whether or not the target person has a cold, etc.); and information on the living environment (temperature, humidity, weather, noise level, and the like) of target person. The life information may be information supplied to the storage device 4 from various systems such as a management system for managing the amount of exercise, schedule, health, and the like of the target person. For example, the attribute/life information storage unit 41 stores the attribute information or/and the life information for each target person in association with the identification information of the target person (subject ID).

The calculation result storage unit 42 stores various calculation results calculated by the information processing device 1. The calculation result storage unit 42 stores, for example, a stress estimate value of the target person calculated by the information processing device 1, the amount of exercise of the target person, and an index relating to stress release of the target person to be described later, in association with the identification information of the target person and the date and time information representing the target date and time. The “target date and time” described above may be the generation date and time of the signal used for calculation, or may be the date and time of the calculation.

The storage device 4 is not limited to the above-described example, and may store various kinds of information necessary for the process executed by the information processing device 1. For example, the storage device 4 may store parameters for configuring various calculation models. Examples of the above-described calculation models include a stress estimation model for the information processing device 1 to estimate the stress value from observation information, an amount-of-exercise calculation model for calculating the amount of exercise the observation information, and an index calculation model for calculating an index, which will be described later, relating to stress release from the estimated stress value (also referred to as “stress estimate value”) and the amount of exercise. Such models may be any machine learning models (including statistical models) such as neural networks, support vector machines or may be predetermined calculation formulas or look-up tables. For example, when the above-described models are models based on neural networks such as a convolution neural network, information regarding various parameters such as a layer structure, a neuron structure of each layer, the number of filters and the size of filters in each layer, and the weight for each element of each filter is stored in the storage device 4.

The configuration of the stress release detection system 100 shown in FIG. 1 is an example, and various changes may be made to the configuration. For example, the input device 2 and the output device 3 may be configured integrally. In this case, the input device 2 and the output device 3 may be configured as a tablet type terminal that is integrated with or separate from the information processing device 1. Further, the input device 2 and the sensor 5 may be configured integrally. Further, the information processing device 1 may be configured by a plurality of devices. In this case, the plurality of devices constituting the information processing device 1 performs transmission and reception of information necessary for executing preassigned processing among the plurality of devices. In this case, the information processing device 1 functions as an information processing system.

### (2) Hardware Configuration of Information Processing Device

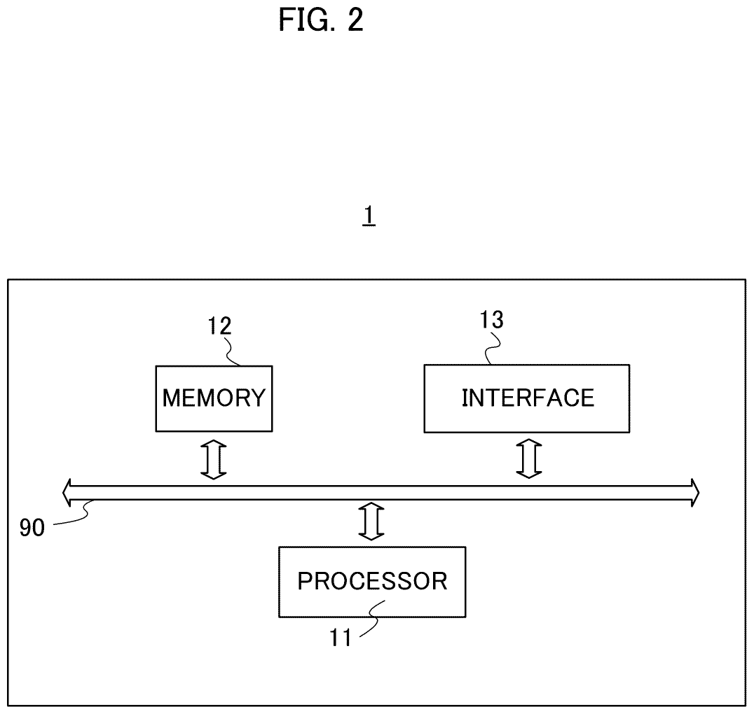

FIG. 2 shows a hardware configuration of the information processing device 1. The information processing device 1 includes a processor 11, a memory 12, and an interface 13 as hardware. The processor 11, memory 12 and interface 13 are connected to one another via a data bus 90.

The processor 11 functions as a controller (arithmetic unit) which controls the entire information processing unit 1 by executing a program stored in the memory 12. Examples of the processor 11 include a CPU (Central Processing Unit), a GPU (Graphics Processing Unit), and a TPU (Tensor Processing Unit). The processor 11 may be configured by a plurality of processors. The processor 11 is an example of a computer.

The memory 12 comprises a variety of volatile and non-volatile memories, such as a RAM (Random Access Memory), a ROM (Read Only Memory), and a flash memory. Further, a program to be executed by the information processing device 1 is stored in the memory 12. A part of the information stored in the memory 12 may be stored in one or more external storage devices that can communicate with the information processing device 1, or may be stored in a removable storage medium detachable from the information processing device 1.

The interface 13 is one or more interfaces for electrically connecting the information processing device 1 to other devices. Examples of these interfaces include a wireless interface, such as a network adapter, for transmitting and receiving data to and from other devices wirelessly, and a hardware interface, such as a cable, for connecting to other devices.

The hardware configuration of the information processing device 1 is not limited to the configuration shown in FIG. 2. For example, the information processing device 1 may include at least one of the input device 2 and/or the output device 3. Further, the information processing device 1 may be connected to or incorporate an audio output device such as a speaker.

### (3) Stress Release Action Detection Process

Next, a description will be given of a stress release action detection process executed by the information processing device 1. In summary, the information processing device 1 calculates an index (also referred to as “stress release index SR”) for making a determination of a stress release action based on the stress estimate value and the amount of exercise regarding the target person, and detects the stress release action based on the stress release index SR. Thus, the information processing device 1 detects the stress release action of the target person with high accuracy and presents the detection result.

### (3-1) Functional Blocks

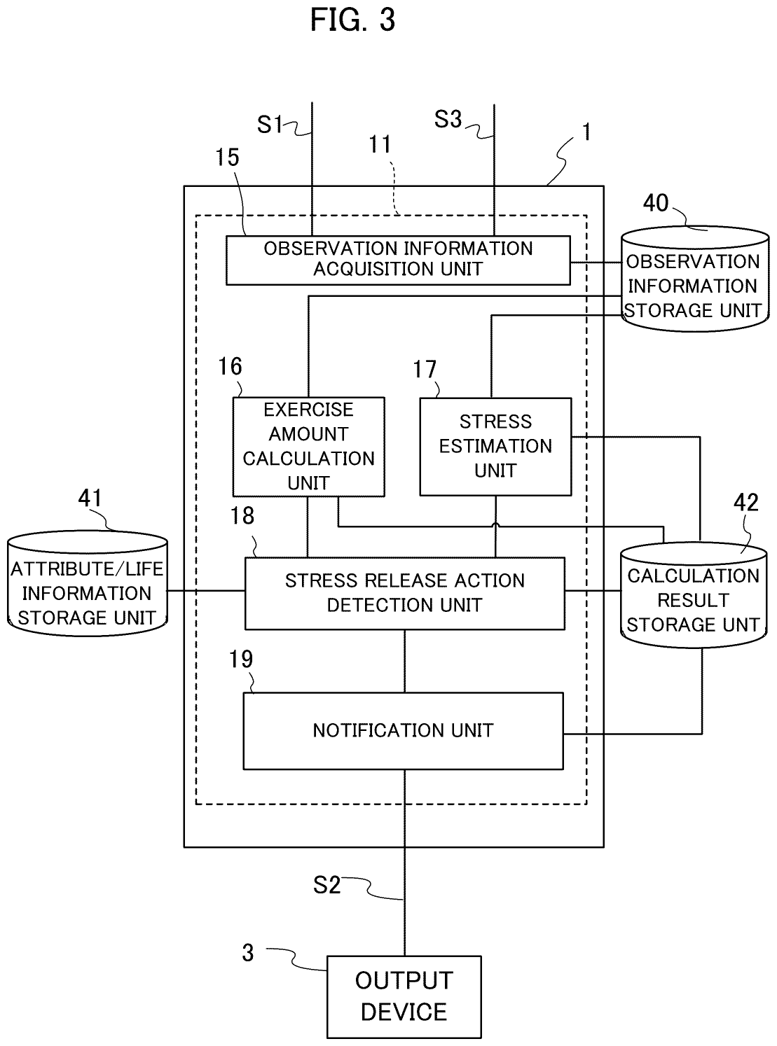

FIG. 3 is an example of functional blocks of the information processing device 1. The processor 11 of the information processing device 1 functionally includes an observation information acquisition unit 15, an exercise amount calculation unit 16, a stress estimation unit 17, a stress release action detection unit 18, and a notification unit 19. In FIG. 3, blocks to exchange data with each other are connected by a solid line, but the combination of blocks to exchange data with each other is not limited to FIG. 3. The same applies to the drawings of other functional blocks described below.

Based on the input signal S1 and the sensor signal S3, the observation information acquisition unit 15 acquires the observation information regarding the target person and stores the acquired observation information in the observation information storage unit 40. In this instance, as described above, the observation information acquisition unit 15 may acquire the sensor signal S3 as the observation information, or may acquire the features calculated based on the sensor signal S3 (including an index representing the facial expression, emotion, and the like analyzed from the image or audio data) as the observation information. Further, the observation information acquisition unit 15 may acquire questionnaire answer information based on the input signal S1 or the diagnostic result regarding the personality or the like based on the questionnaire answer information as the observation information.

The exercise amount calculation unit 16 calculates, based on the observation information stored in the observation information storage unit 40, the amount of exercise of the target person (more specifically amount of exercise per unit time). In this case, the exercise amount calculation unit 16 may use, as the observation information to be used for the exercise amount calculation, biometric information (e.g., the amount of variation in acceleration, the amount of increase in heart rate, the amount of increase in body temperature, or/and the amount of increase in skin temperature) obtained from a wearable terminal or the like worn by the target person during the exercise amount calculation or may use device information (e.g., the amount of change in acceleration, the amount of change in the position measured by GNSS) obtained from a smartphone or the like possessed by the target person. In this case, for example, the exercise amount calculation unit 16 inputs the above-mentioned observation information to an exercise amount calculation model whose parameters are stored in advance in the storage device 4 and acquires the amount of exercise outputted by the exercise amount calculation model in response to the input. In this case, the exercise amount calculation model is a model configured to output the amount of exercise when a predetermined type of observation information is inputted thereto.

It is noted that the exercise amount calculation model is not limited to the learned model. For example, the exercise amount calculation model may be configured to output an average value per unit time of the norm of the acceleration vectors when acceleration vectors outputted in time series by a triaxial acceleration sensor is inputted to the exercise amount calculation model. In another example, the exercise amount calculation model may be configured to classify a motion state (e.g., the walking state and the running state) based on the periodic change in the accelerations outputted by an acceleration sensor and then output the amount of exercise according to the classification result. The exercise amount calculation unit 16 supplies the calculated amount of exercise to the stress release action detection unit 18 and stores it in the calculation result storage unit 42.

The stress estimation unit 17 calculates the stress estimate value of the target person based on the observation information stored in the observation information storage unit 40. In this case, the stress estimation unit 17 may use any information having a correlation with stress (e.g., biological information regarding heartbeat, perspiration, and/or skin temperature, facial expression/emotion information recognized from an image or a voice, a questionnaire result, a personality diagnosis result, and/or an operation log) as the observation information used for calculating the stress estimate value. In this case, for example, the stress estimation unit 17 inputs the above-described observation information to a stress estimation model whose parameters are stored in the storage device 4 in advance and thereby acquires the stress estimate value that is a stress value outputted by the stress estimation model in response to the input.

It is noted that the stress estimation model is not limited to the learned model. For example, the stress estimation model may be an expression for deriving a stress estimate value from the degree of fluctuation of the heart rate per unit time, the number of peaks in the amount of perspiration per unit time, and the like. The stress estimation unit 17 supplies the calculated stress estimate value to the stress release action detection unit 18 and stores it in the calculation result storage unit 42.

The stress release action detection unit 18 calculates the stress release index SR based on the amount of exercise of the target person supplied from the exercise amount calculation unit 16 and the stress estimate value of the target person supplied from the stress estimation unit 17, and detects the stress release action based on the stress release index SR. Hereafter, for explanatory convenience, the stress release index SR is assumed to be an index value that increases with an increase in the degree of stress release, as an example. Alternatively, the stress release action detection unit 18 may calculate such an index value that decreases with an increase in the degree of stress release.

The stress release action detection unit 18 calculates the stress release index SR as an index value that increases with an increase in the amount of exercise (i.e., has a positive correlation with the amount of exercise) and decreases with an increase in the stress estimate value (i.e., has a negative correlation with the stress estimate value). As a typical example, the stress release action detection unit 18 calculates the stress release index SR by the following equation (1) when the amount of exercise is denoted by “M” and the stress estimate is denoted by “S”.

$$\\ \begin{matrix}SR = M / S & ( 1 )\end{matrix}$$

Then, for example, if the stress release index SR is equal to or larger than a predetermined lower limit threshold value (also referred to as “lower limit threshold value Th1”), the stress release action detection unit 18 determines that a stress release action is detected.

In some embodiments, the stress release action detection unit 18 sets, in addition to the lower limit threshold value Th1, an upper limit threshold value (also referred to as “upper limit threshold value Th2”) for the stress release index SR. Then, if the stress release index SR is equal to or larger than the lower limit threshold value Th1 and less than the upper limit threshold value Th2, the stress release action detection unit 18 determines that a stress release action is detected. As will be described later, when the stress release index SR is extremely high, the target person is being moved by a vehicle or the like, and there is a high possibility that the actual stress release of the target person is not reflected in the stress release index SR. Taking the above into consideration, if the stress release index SR is equal to or greater than the upper limit threshold value Th2, the stress release action detection unit 18 does not determine that a stress release action is detected. Each of the lower limit threshold value Th1 and the upper limit threshold value Th2 is set to a fitting value which is previously stored, for example, in the storage device 4 or the like.

In some embodiments, the stress release action detection unit 18 may refer to at least one of the attribute information and/or the life information regarding the target person stored in the attribute/life information storage unit 41 and determine the stress release index SR in further consideration of these information. This specific example will be described later. The stress release action detection unit 18 supplies the detection result of the stress release action to the notification unit 19. The stress release action detection unit 18 stores the calculated stress release index SR or the like in the calculation result storage unit 42.

The notification unit 19 controls the output device 3 to output information regarding the stress release action based on the detection result of the stress release action supplied from the stress release action detection unit 18 and the information stored in the calculation result storage unit 42. An example of the output control by the notification unit 19 will be specifically described in the section of “(3-4) Notification Example”.

Here, for example, each component of the observation information acquisition unit 15, the exercise amount calculation unit 16, the stress estimation unit 17, the stress release action detection unit 18 and the notification unit 19 described in FIG. 3 can be realized by the processor 11 executing a program. In addition, the necessary program may be recorded in any non-volatile storage medium and installed as necessary to realize the respective components. In addition, at least a part of these components is not limited to being realized by a software program and may be realized by any combination of hardware, firmware, and software. At least some of these components may also be implemented using user-programmable integrated circuitry, such as FPGA (Field-Programmable Gate Array) and microcontrollers. In this case, the integrated circuit may be used to realize a program for configuring each of the above-described components. Further, at least a part of the components may be configured by an ASSP (Application Specific Standard Produce), an ASIC (Application Specific Integrated Circuit) and/or a quantum processor (quantum computer control chip). In this way, each component may be implemented by a variety of hardware. The above is true for other example embodiments to be described later. Further, each of these components may be realized by the collaboration of a plurality of computers, for example, using cloud computing technology.

(3-2) Relation between Stress Release Action and Index

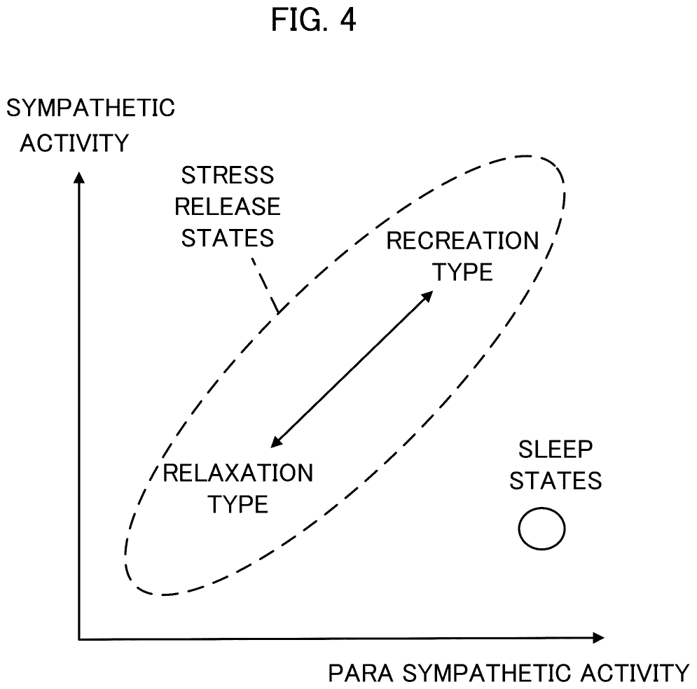

Next, a supplementary description will be given of the relation between stress release action and stress release index SR. FIG. 4 shows a two-dimensional map with sympathetic activity on the vertical axis and parasympathetic activity on the horizontal axis. In the two-dimensional map shown in FIG. 4, a region corresponding to stress release states is indicated by dashed oval and a region corresponding to sleep states in indicated by a solid circle.

As shown in FIG. 4, a stress release state occurs when the degree of parasympathetic activity is in proportional to the degree of sympathetic activity. The stress release is roughly classified into two types, the recreation type and the relaxation type. Here, the recreation type is stress release with physical activity, and it is caused by actions such as exercise, karaoke, and patrol in a theme park and trip. The relaxation type is stress release without physical activity, and occurs by actions such as music listening, sitting, meditation, forest bathing, natural bathing, aroma, and deep breathing. Then, in the present example embodiment, the information processing device 1 detects the stress release state including these types.

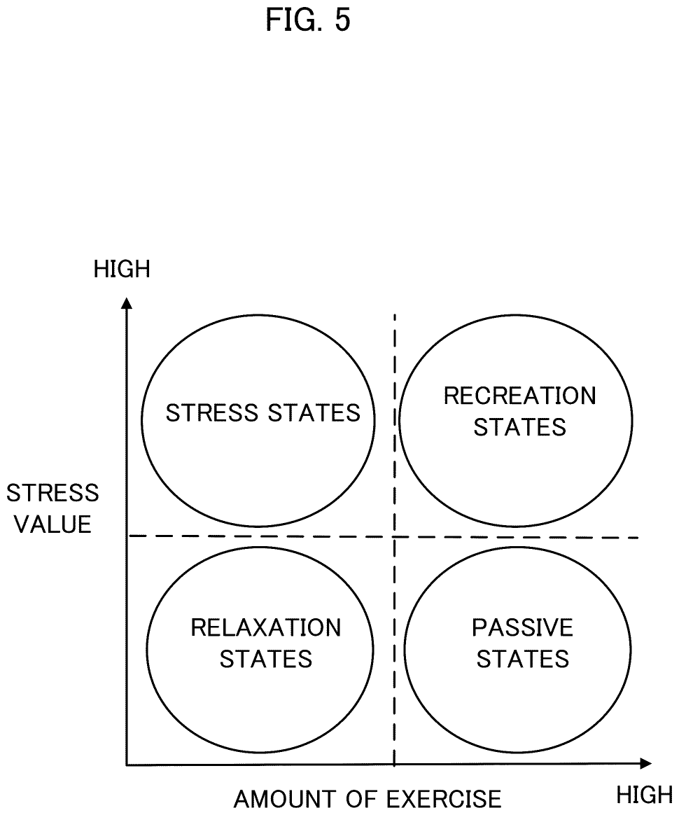

FIG. 5 shows a two-dimensional map with the stress value on the vertical axis and amount of exercise on the horizontal axis. In the two-dimensional map shown in FIG. 5, there are clear indications of a region corresponding to “stress states” in which the target person feels stress, a region corresponding to “relaxation states” in which the target person feels relaxed, a region corresponding to “recreation states” in which the target person is recreated, a region corresponding to “passive states” in which the target person is moving in a vehicle or the like. Roughly, “stress states” are states where the stress value is a high stress value higher than a threshold value and the amount of exercise is lower than a threshold value, and “relaxation states” are states where the stress value is a low stress value that is lower than the threshold value and the amount of exercise is lower than the threshold value. The “recreation states” are states where the stress value is the high stress value and the amount of exercise is higher than the threshold value, and the “passive states” are states where the stress value is the low stress value and the amount of exercise is high amount of exercise (in other words, the states in which the amount of exercise of the target person has increased due to passive activity). It should be noted that the stress value is proportional to the sympathetic activity adopted as the vertical axis of the two-dimensional map shown in FIG. 4.

As shown in FIG. 5, “relaxation states” and “recreation states” corresponding to the stress release states are equivalent to a state where the stress value and the amount of exercise are both low or both high. Therefore, in the case of the stress release index SR based on the definition of the equation (1), the stress release state corresponds to the case where the stress release index SR is an intermediate value range excluding a value range that is too high (i.e., a value range that is larger than or equal to the upper limit threshold value Th2) and a value range that is too low (i.e., a value range that is less than the lower limit threshold value Th1).

In general, “passive states” shown in FIG. 5 do not basically occur because the higher the amount of exercise of the target person is, the higher the amount of sympathetic activity (i.e., the stress value) of the target person becomes. On the other hand, when the target person is being moved on a vehicle or the like, the apparent value of the acceleration of the target person detected by the sensor 5 increases, and the amount of exercise is calculated to be substantially higher, resulting in a state (i.e., a passive state) where the amount of exercise is high and the stress value is low. Such a state is a state that occurs due to false measurement (i.e., erroneous measurement) of the acceleration of the target person by the sensor 5.

Taking the above into consideration, in such a case where the stress release action corresponds to the above-mentioned passive state (that is, the state in which the stress release index SR is equal to or more than the upper limit threshold value Th2), the stress release action detection unit 18 according to the present example embodiment determines, by using the stress value, that the stress release action is detected, if the stress value is equal to or smaller than a threshold value “Th_s”. The threshold value Th_s is set to a fitting value previously stored in the storage device 4 or the like, for example. For example, when the target person is a driver riding in a vehicle, the stress release action detection unit 18 determines that the passive state is detected if the stress release index SR is equal to or larger than the upper limit threshold value Th2 due to the high amount of exercise, and then it determines that a stress release action is detected if the stress value of the target person is equal to or less than the threshold value Th_s. The same applies to the case where the target person is a person riding on the passenger seat or a person traveling by train.

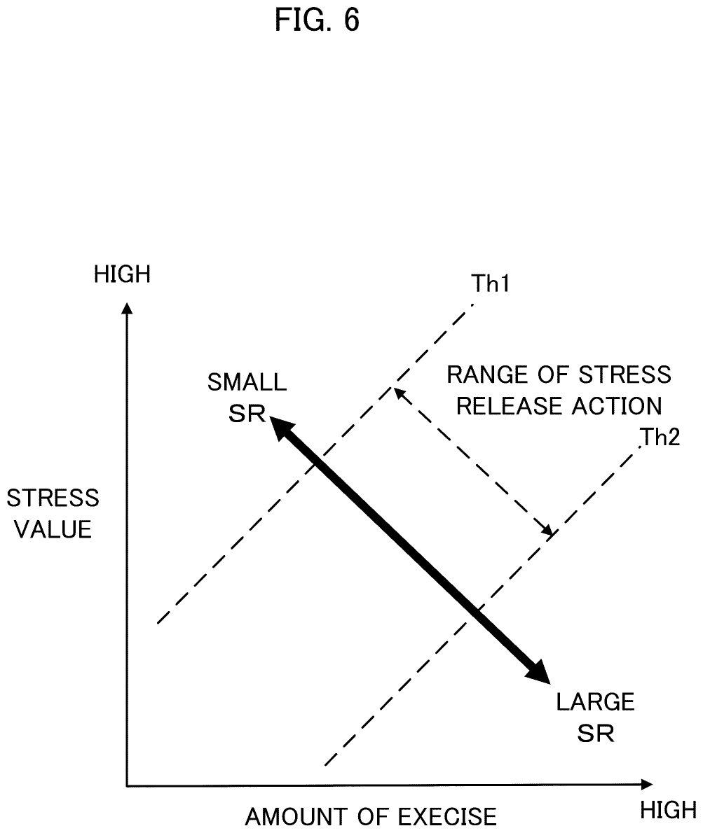

FIG. 6 shows a two-dimensional map with the stress value on the vertical axis and amount of exercise on the horizontal axis with a schematic visualization of the stress release index SR based on the equation (1) and the lower limit threshold value Th1 and the upper limit threshold value Th2.

As shown in FIG. 6, the stress release index SR decreases with an increase in the stress value and/or with a decrease in the amount of exercise whereas the stress release index SR increases with a decrease in the stress value and/or with an increase in the amount of exercise. In the present example embodiment, if the stress release index SR is equal to or greater than the lower limit threshold value Th1 and less than the upper limit threshold value Th2, the stress release action detection unit 18 determines that a stress release action is detected. In this case, the range recognized as a stress release action determined by the lower limit threshold value Th1 and the upper limit threshold value Th2 is the range to which “relaxation states” and “recreation states” shown in FIG. 5 belong. Thus, the stress release action detection unit 18 can suitably detect the stress release action corresponding to the “relaxation states” or “recreation states”.

According to the method of detecting the stress release action by the stress release index SR described above, the stress release action detection unit 18 can suitably detect stress release based on the stress release action such as listening and exercise in daily life. The stress release action detection unit 18 can also detect stress release caused by a physical activity or a stress release method (movement, karaoke, etc.) which could come with an excited state.

### (3-3) Index Calculation Using Attribute Information and Life Information

The stress release action detection unit 18 may determine the stress release index SR based on at least one of the attribution information and/or the living information regarding the target person.

First, a description will be given of a case in which attribute information is used. For example, based on the attribute of the target person, the stress release action detection unit 18 switches the index calculation model for use in calculating the stress release index SR. For example, if the attribute information includes information as to whether the target person likes or dislikes exercises, the stress release action detection unit 18 determines that an increase in amount of exercise is particularly effective for stress release if the target person likes exercises. Therefore, in this case, for example, the stress release action detection unit 18 calculates the stress release index SR according to the following equation (2) (M=amount of exercise, S=stress estimate value) using a correction coefficient α greater than 1 as an exponential coefficient of amount of exercise.

$$\\ \begin{matrix}SR = {M}^{{}_{} α} / S & ( 2 )\end{matrix}$$

On the other hand, if the attribute information indicates that the target person dislikes exercises, the stress release action detection unit 18 determines that an increase in the amount of exercise is not effective for the stress release and then calculates the stress release index SR according to the above-described equation (2) in which the correction coefficient α is set to be smaller than 1. In this way, in the case of the target person who likes exercises, the increase in the amount of exercise is likely to lead to an increase in the stress release index SR, whereas in the case of the target person who dislikes exercises, an increase in the amount of exercise is less likely to lead to an increase in the stress release index SR.

Similarly, when the attribute information indicates another type of the attribute such as an age and gender, the stress release action detection unit 18 prepares index calculation models configured to calculate an appropriate stress release index SR for categories (e.g., age-specific categories, gender-specific categories) of the attribute, and switches the index calculation model to be used according to the attribute of the target person. The parameters (the value of a in the equation (2)) for configuring each index calculation model are stored in advance in the storage device 4 or the like in association with each category of the attribute to be classified. Thus, the stress release action detection unit 18 can more accurately calculate the stress release index SR considering the attribute of the target person.

Next, a description will be given of the case in which the life information is used. For example, the stress release action detection unit 18 switches the index calculation model for use in calculating the stress release index SR, based on the living information regarding the target person. For example, if the life information is schedule information indicating whether or not the target person is working, the stress release action detection unit 18 recognizes whether or not the target person is working at the target date and time of determination of the stress release action, and calculates the stress release index SR by using the index calculation model selected according to the recognition result. In this case, an index calculation model to be used to determine the stress release action during the working period of the target person and another index calculation model to be used to determine the stress release action during the non-working period are prepared in advance, respectively. Similarly, for any other life information, index calculation models are prepared in advance for respective life patterns to be identified by the life information, and the stress release action detection unit 18 selects an index calculation model corresponding to a life pattern of the target person in the target period identified by referring to the life information regarding the target person to thereby calculate the stress release index SR.

When the target person is working, noises during work (e.g., the sound of a tool or a heavy machine) also affects the presence or absence of stress release. Thus, in calculating the stress release index SR to be used for determining the stress release action during the work, the stress release action detection unit 18 may further refer to the information indicating a noise level at the work field. Such a noise level may be specified by a sensor signal S3 outputted by the sensor 5 such as a noise sensor, for example. In addition, if the stress release action detection unit 18 determines, with reference to occupational information included in the attribution information, that the target person is mainly engaged in a physical task such as a sports player, the stress release action detection unit 18 determines that the working time is not a target period of determination of the stress release action. Thus, the stress release action detection unit 18 may not calculate the stress release index SR of the target person during the work.

Further, when the information regarding the daily amount of exercise which is the average amount of exercise performed by the target person is further included in the life information, the stress release action detection unit 18 may normalize the amount of exercise calculated by the exercise amount calculation unit 16 by the daily amount of exercise. In this case, for example, the stress release action detection unit 18 calculates the normalized amount of exercise (i.e., “calculated amount of exercise” x “average amount of exercise”/“daily amount of exercise”) obtained by dividing the amount of exercise calculated by the exercise amount calculation unit 16 by the ratio (i.e., “daily amount of exercise”/“average amount of exercise”) of the daily amount of exercise to the average amount of exercise of the general person (average amount of exercise). The stress release action detection unit 18 calculates the stress release index SR by using the normalized amount of exercise calculated in such a way, suitably calculating the stress release index SR taking into account the difference in daily exercise amounts of individuals.

The stress release action detection unit 18 may change at least one of the lower limit threshold value Th1 and/or the upper limit threshold value Th2 based on the daily amount of exercise, in addition to or in place of the process of normalizing the amount of exercise calculated by the exercise amount calculation unit 16 based on the daily amount of exercise. In this instance, the stress release action detection unit 18 increases the lower limit threshold value Th1 with an increase in the daily amount of exercise. Thus, the stress release action detection unit 18 can accurately determine whether or not the stress release action is performed even for a person having a high daily amount of exercise such as a sports player and a physical worker.

### (3-4) Notification Example

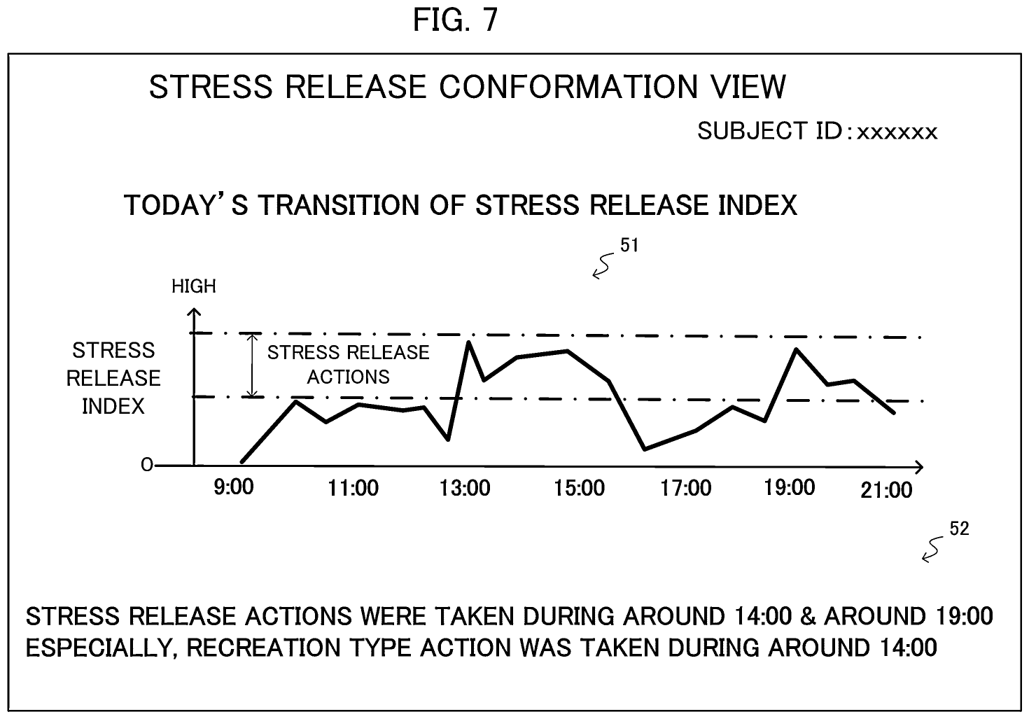

FIG. 7 is an example of a stress release confirmation screen image which the notification unit 19 displays on the output device 3. Here, as an example, the notification unit 19 includes an index graph display area 51 representing a transition of the stress release index SR of a target person on a date (today in this example) specified by the user and a text information display area 52 representing a text sentence indicative of a period of time when the stress release action is detected on the stress release confirmation screen image. The notification unit 19 generates an output control signal S2 for displaying the stress release confirmation screen image, and supplies the output control signal S2 to the output device 3 via the interface 13, thereby causing the output device 3 to display the stress release confirmation screen image.

In the example shown in FIG. 7, the exercise amount calculation unit 16 and the stress estimation unit 17 calculates the amounts of exercise and the stress estimate values in the time series based on the observation information generated in time series in the target date. The exercise amount calculation unit 16 and the stress estimation unit 17 may sequentially calculate the amount of exercise and the stress estimate value based on the observation information every time the observation information acquisition unit 15 acquires the observation information, or may collectively calculate the amounts of exercise and the stress estimate values based on the observation information upon receiving a display request of the stress release confirmation screen image. Then, the stress release action detection unit 18 calculates values of the stress release index SR in time series in the target day based on the calculated amounts of exercise and stress estimate values in time series in the target day.

The notification unit 19 displays, in the index graph display area 51, a graph of the transition of the stress release index SR of the target person on the two-dimensional coordinates with the time on the horizontal axis and the stress release index SR on the vertical axis. Further, the notification unit 19 clearly indicates, in the graph described above, the lower limit threshold value Th1 and the upper limit threshold value Th2 by dashed lines, thereby clarifying the range which is determined to be stress release actions on the index graph display area 51.

Further, based on the detection result from the stress release action detection unit 18, the notification unit 19 recognizes periods of time (in this case around 14 o'clock and 19 o'clock) when the stress release action is detected and a period of time (in this case around 14 o'clock), among the periods of time, when an action which belongs to the recreation type is detected, respectively. Thus, the notification unit 19 displays text information regarding these periods of time in the information display area 52. As shown in FIG. 5, the stress value and amount of exercise are different between the relaxation type and the recreation type, respectively. Thus, for example, if a stress release action is detected, and, if the corresponding amount of exercise is equal to or more than a predetermined threshold value and/or the corresponding stress estimate value is equal to or more than a predetermined threshold value, the stress release action detection unit 18 or the notification unit 19 determines that an action which belongs to the recreation type has been performed. For example, the above threshold values are stored in advance in the storage device 4.

Thus, according to the display example shown in FIG. 7, the notification unit 19 can suitably notify the target person or the manager of the timing at which the stress release action was performed, the timing at which there was an action which belongs to the recreation type, or the like. Then, the target person or manager can use the reported information to manage the stress of the target person or to maintain the motivation for continuation of stress management of the target person. Besides, if the target person is an employee, the manager can also use the information to manage the mental health of the employee in the workplace and assign work.

The aspect of the notification of the stress release action is not limited to the display example shown in FIG. 7. In the first example, the notification unit 19 may output the detection result of the stress release action of the target person in a weekly or monthly unit (i.e., in any predetermined period specified by the user). In this case, for example, the notification unit 19 may output information regarding the statistical tendency of the period of time when a stress release action (may be limited to an action which belongs to the recreation type, hereinafter the same.) occurs. In another example, the notification unit 19 may output information regarding a statistical tendency regarding the types (the recreation type, relaxation type) of stress release action. Thus, the notification unit 19 can suitably notify the user that what kind of stress release actions the target person is likely to take (e.g., whether the target person tends to take an action which belongs to the relaxation type or an action which belongs to the recreation type).

In the second example, the notification unit 19 may output the detection result of the stress release action or the like in accordance with the current state of the target person in real time. In this case, the information processing device 1 determines the presence or absence of the stress release action using the observation information based on the sensor signal S3 obtained in real time. Then, if a stress release action is detected, the information processing device 1 immediately causes the output device 3 to notify the user that the stress release action has been performed by the output device 3. In this instance, the exercise amount calculation unit 16 and the stress estimation unit 17 calculates the amount of exercise and the stress estimate value for the observation information representing the current state of the target person acquired by the observation information acquisition unit 15, and the stress release action detection unit 18 calculates the stress release index SR based on the amount of exercise and the stress estimate value to make a determination of the stress release action. According to the second example, the notification unit 19 suitably notifies the target person of the occurrence of the stress release action, thereby urging the target person to take an action leading to stress relief.

In the third example, the notification unit 19 may output the detection result of the stress release action or the like at any time after it is determined that a stress release action is detected. In the fourth example, the notification unit 19 may notify the user of the presence or absence of a stress release action, instead of notifying the user of the degree of stress release as in the example shown in FIG. 7. In this case, the notification unit 19 may notify by audio (including voice) the user that there was a stress release action, or may notify by display the user that there was a stress release action.

### (3-5) Processing Flow

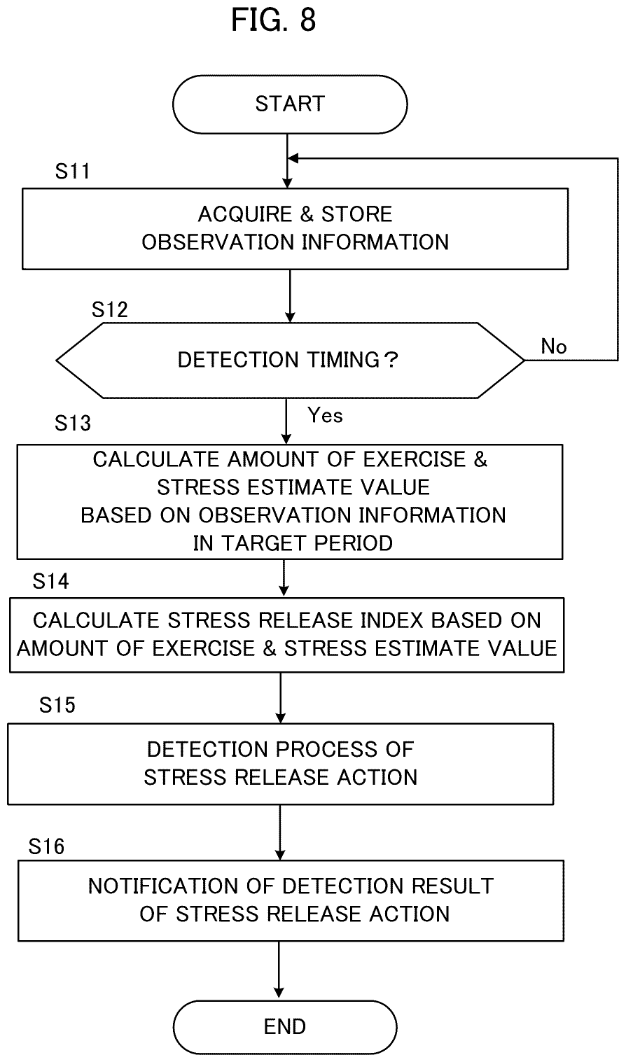

FIG. 8 is an example of a flowchart that the information processing device 1 executes in the first example embodiment. For example, the information processing device 1 executes the process of the flowchart shown in FIG. 8, if it is determined to be a predetermined stress estimation timing.

First, the information processing device 1 acquires observation information on the basis of the input signal S1 supplied from the input device 2 or/and the sensor signal S3 supplied from the sensor 5, and stores the acquired observation information in the observation information storage unit 40 (step S11).

Then, the information processing device 1 determines whether or not it is the detection timing of the stress release action (step S12). Then, if it is the detection timing of the stress release action (step S12; Yes), the information processing device 1 proceeds with the process at step S13. On the other hand, if it is not the detection timing of the stress release action (step S12; No), the information processing device 1 gets back to the process at step S11.

Next, at step S13, the information processing device 1 calculates the amount of exercise and the stress estimate value of the target person based on the observation information in the target period of the detection of the stress release action (step S13). Then, the information processing device 1 calculates the stress release index SR based on the amount of exercise and the stress estimate value calculated at step S13 (step S14). Then, the information processing device 1 performs detection process of the stress release action of the target person on the basis of the calculated stress release index SR (step S15). In this case, for example, based on the stress release index SR and the lower limit threshold value Th1 and the upper limit threshold value Th2, the information processing device 1 determines the presence or absence of the stress release action. Then, the information processing device 1 performs the process of making a notification of the detection result of the stress release action generated at step S15 (step S16).

### (4) Modifications

Next, a description will be given of modifications of the example embodiment described above. The following modifications may be applied to the example embodiments described above in any combination.

### (First Modification)

The stress release action detection unit 18 may perform a detection process that is specialized for an action which belongs to the recreation type among the stress release actions.

In this case, if the stress release action is detected and the amount of exercise and/or the stress estimate value is determined to be equal to or larger than the corresponding predetermined threshold value, the stress release action detection unit 18 determines that an action which belongs to the recreation type has been performed. Then, the notification unit 19 makes a notification process of the detection result of the action which belongs to the recreation type, based on the detection result from the stress release action detection unit 18. Thus, the information processing device 1 can suitably notify the target person or the manager of the timing or the like in which an action which belongs to the recreation type comes up.

### (Second Modification)

In determining the presence or absence of the stress release action, the stress release action detection unit 18 may determine the presence or absence of the stress release action based on not only the stress release index SR and the threshold value but also the stress value obtained after a lapse of a predetermined time.

A description will be given of a specific example regarding a determination of the presence or absence of the stress release action at 13 o'clock in the case shown in FIG. 7. In this case, if the stress release index SR at 13 o'clock is equal to or larger than the lower limit threshold value Th1 and less than the upper limit threshold value Th2, and, if the stress estimate value obtained after a lapse of a predetermined time (e.g., several minutes or dozens of minutes) from 13 o'clock has decreased by a predetermined value or a predetermined rate or more compared to the stress estimation value at 13 o'clock, the stress release action detection unit 18 determines that there is a stress release action at 13 o'clock. The predetermined value or a predetermined rate described above is stored in advance in the storage device 4 or the like, for example.

According to the present modification, the stress release action detection unit 18 can determine the presence or absence of the stress release action with more accuracy in consideration of the transition of the actual stress value obtained after the target timing of the determination of the stress release action.

### (Third Modification)

The stress release action detection unit 18 may determine that a stress release action is detected if a criterion for the stress release index SR is continuously met.

In this case, for example, for each period (time window) having a predetermined time length, the stress release action detection unit 18 aggregates the determination results of the presence or absence of the stress release action based on the stress release index SR calculated at respective calculation timings. Then, the stress release action detection unit 18 determines the presence or absence of the stress release action for each time window, based on the aggregation result. For example, when the time window is 10 minutes and the stress release index SR is calculated every 30 seconds, the stress release action detection unit 18 determines whether or not each of 20 values of the stress release index SR calculated in a time window of interest satisfies the criterion regarding the stress release action (i.e., the criterion in which the lower limit threshold value Th1 and the upper limit threshold value Th2 are used). Then, if a predetermined number or more (e.g., more than half) of the 20 values of the stress release index SR satisfy the criterion regarding the stress release action, the stress release action detection unit 18 determines that a stress release action is detected in the time window of interest. The stress release action detection unit 18 determines whether or not each value of the stress release index SR calculated in the time window of interest satisfies the criterion regarding the stress release action (i.e., the criterion in which the lower limit threshold value Th1 and the upper limit threshold value Th2 are used) and determines that the stress release action is continued if the duration of time when the criterion regarding the stress release action is satisfied exceeds a predetermined determination time length. In this case, for example, the stress release action detection unit 18 determines that the stress release action is continued upon detecting the stress release action continuously for 5 minutes.

According to this modification, the information processing device 1 can more stably detect the presence or absence of the stress release action.

### (Fourth Modification)

The stress release action detection unit 18 may perform the detection of the stress release action by further considering temporal variation in the stress release index SR.

The stress release action detection unit 18 determines that the stress release action is started, if the criterion regarding the stress release index SR (that is, a criterion in which the lower limit threshold value Th1 and the upper limit threshold value Th2 are used) is satisfied or/and if an increase rate of the stress release index SR (an increase rate with respect to the stress release index SR calculated immediately before) is equal to or more than a predetermined rate. The stress release action detection unit 18 determines that the stress release action is terminated if the criterion regarding the stress release index SR (that is, the criterion in which the lower limit threshold value Th1 and the upper limit threshold value Th2 are used) is no longer satisfied or/and the descent rate of the stress release index SR (the decreasing rate with respect to the stress release index SR calculated immediately before) is equal to or more than a predetermined rate.

According to this modification, the stress release action detection unit 18 can accurately detect the duration in which the stress release action has been performed based on the variation in the stress release index SR.

### (Fifth Modification)

The stress release action detection unit 18 may determine the stress release index SR based on the subtraction process using the stress estimate value and the amount of exercise, instead of determining the stress release index SR based on the ratio between the amount of exercise and the stress estimate value as shown in the equation (1) or the equation (2). For example, the stress release action detection unit 18 normalizes the stress estimate value and the amount of exercise so as to be in the value range from 0 to 1, respectively. Thereafter, the stress release action detection unit 18 multiplies at least one of the stress estimate value and/or the amount of exercise by a predetermined weight. After the normalization and the multiplication of the weight, the stress release action detection unit 18 calculates the stress release index SR that is a value obtained by subtracting the stress estimate value by the amount of exercise. In this way, the stress release index SR may be determined by any calculation method using stress estimate value and the amount of exercise.

### (Sixth Modification)

The information processing device 1 may detect the stress release action without using the amount of exercise and notify the target person or the manager of the detection result.

In this case, if the stress estimation value calculated by the stress estimation unit 17 is equal to or less than a predetermined threshold value or if the descent rate of the stress estimation value is equal to or more than a predetermined rate, the information processing device 1 determines that a stress release action is detected. Then, the information processing device 1 notifies the target person or the manager of the detection result of the stress release action. Thus, the information processing device 1 can suitably provide information that can be used for the stress management of the target person or the motivation maintenance to the stress management continuation to the target person or the manager.

### Second Example Embodiment

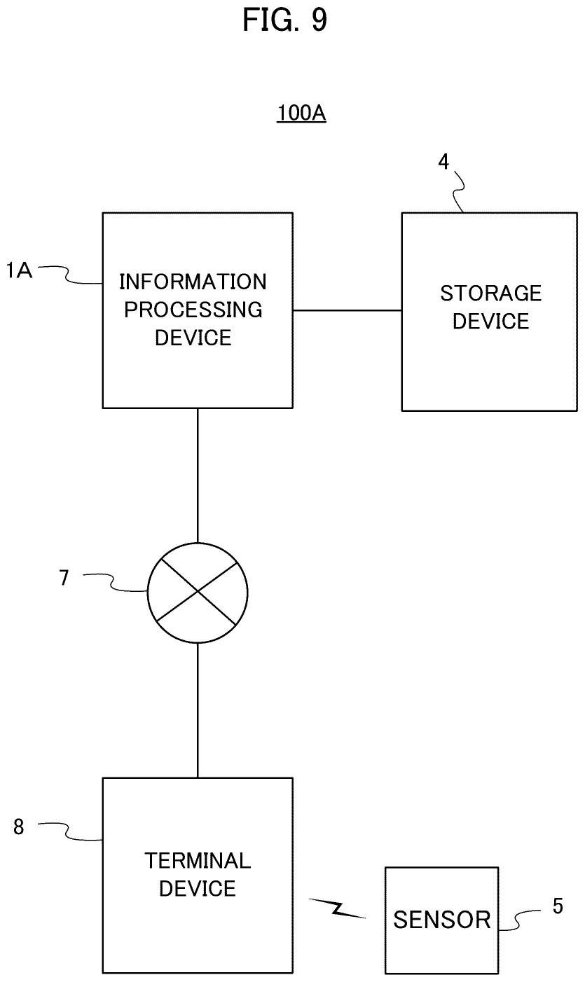

FIG. 9 shows a schematic configuration of a stress release detection 100A according to the second example embodiment. The stress release detection system 100A according to the second example embodiment is a system of a server client model, and an information processing device 1A functioning as a server device executes the processes to be executed by the information processing device 1 according to the first example embodiment. Hereinafter, the same components as those in the first example embodiment are appropriately denoted by the same reference numerals, and a description thereof will be omitted.

As shown in FIG. 9, the stress release detection system 100A mainly includes an information processing device 1A functioning as a server, a storage device 4, and a terminal device 8 functioning as a client. The information processing device 1A and the terminal device 8 perform data communication with each other via the network 7.

The terminal device 8 is a terminal used by the user serving as a target person, and is equipped with an input function, display function, and communication function, and functions as the input device 2 and the output device 3 shown in FIG. 1. The terminal device 8 may be, for example, a personal computer, a tablet-type terminal such as a smartphone, a PDA (Personal Digital Assistant), or the like. The terminal device 8 is electrically connected to the sensor 5, such as a wearable sensor worn by the user, and transmits a biological signal or the like (i.e., information equivalent to the sensor signal S3 in FIG. 1) of the target person outputted by the sensor 5 to the information processing device 1A. Further, the terminal device 8 receives the user input or the like relating to the answer of the questionnaire, and transmits information (information equivalent to the input signal S1 in FIG. 1) generated by the user input to the information processing device 1A.

The information processing device 1A is equipped with the same hardware configuration as the hardware configuration of the information processing device 1 shown in FIG. 2, and the processor 11 of the information processing device 1A is equipped with functional blocks shown in FIG. 3. The information processing device 1A receives the information equivalent to the input signal S1 and the sensor signal S3 in FIG. 1 from the terminal device 8 through the network 7 and executes a stress estimation process. The information processing device 1A transmits an output signal for outputting the stress estimation result to the terminal device 8 through the network 7 based on an output request from the terminal device 8.

According to the second example embodiment, the detection of the stress release action of the target person is performed based on the biological signal or the like of the target person received from the terminal used by the target person, and the target person can be suitably notified of the detection result.

### Third Example Embodiment

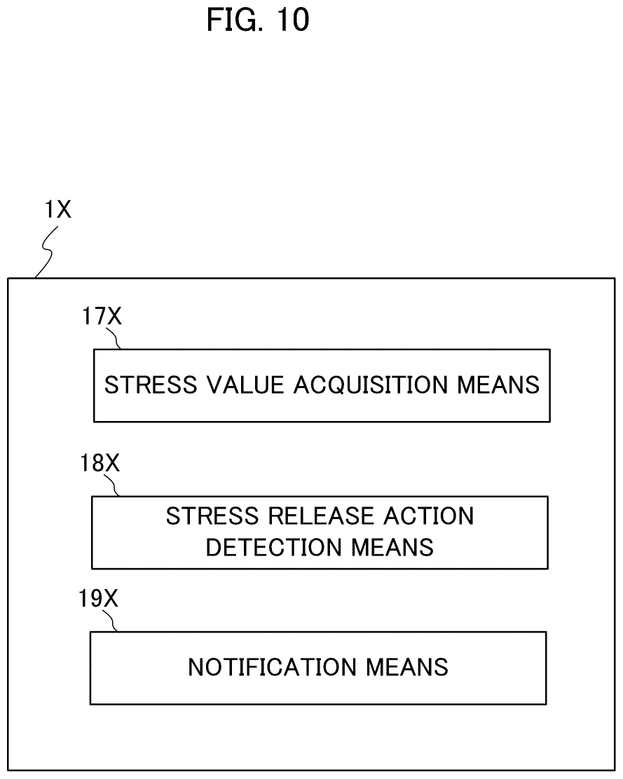

FIG. 10 is a block diagram of an information processing device 1X according to the third example embodiment. The information processing device 1X mainly includes a stress value acquisition means 17X, a stress release action detection means 18X, and a notification means 19X. The information processing device 1X may be configured by a plurality of devices.

The stress value acquisition means 17X is configured to acquire a stress value representing a degree of stress of a target person. The stress value acquisition means 17X may acquire the stress value by estimating the stress value from a biomedical signal or the like of the target person, or may acquire the stress value of the target person stored in the storage device or the like or calculated by another device. Examples of the stress value acquisition means 17X in the former case include a stress estimation unit 17 according to the first example embodiment (including modifications, hereinafter the same) and the second example embodiment.

The stress release action detection means 18X is configured to detect a stress release action, which is an action for releasing a stress, based on the stress value. Examples of the stress release action detection means 18X include the stress release action detection unit 18 according to the first example embodiment and the second example embodiment.

The notification means 19X is configured to make a notification of a detection result of the stress release action. In this case, the notification means 19X may be configured to make a notification of the detection result of the stress release action by audio (including voice), or may be configured to make a notification of the detection result of the stress release action by displaying information. Examples of the notification means 19X include the notification unit 19 in the first example embodiment and the second example embodiment.

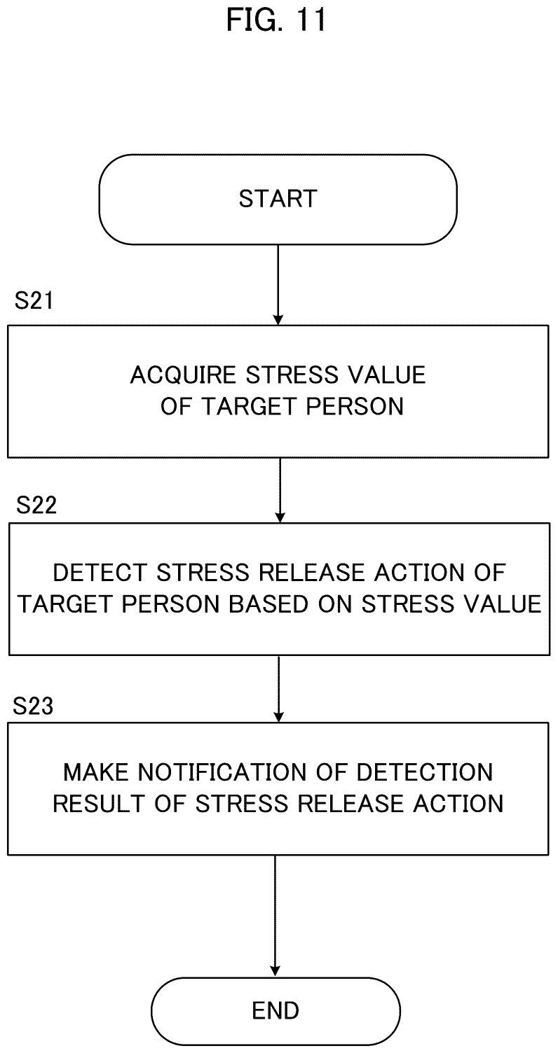

FIG. 11 is an exemplary flowchart that is executed by the information processing device 1X in the third example embodiment. First, the stress value acquisition means 17X acquires a stress value representing a degree of stress of a target person (step S21). Next, the stress release action detection means 18X detects a stress release action, which is an action for releasing a stress, based on the stress value (step S22). Then, the notification means 19X makes a notification of a detection result of the stress release action (step S23).

According to the third example embodiment, the information processing device 1X detects the stress release action and notifies the user of the detection result. Thus, it is possible to suitably provide information that can be used for maintaining the motivation to stress management and continuation of stress management by the target person.

In the example embodiments described above, the program is stored by any type of a non-transitory computer-readable medium (non-transitory computer readable medium) and can be supplied to a control unit or the like that is a computer. The non-transitory computer-readable medium include any type of a tangible storage medium. Examples of the non-transitory computer readable medium include a magnetic storage medium (e.g., a flexible disk, a magnetic tape, a hard disk drive), a magnetic-optical storage medium (e.g., a magnetic optical disk), CD-ROM (Read Only Memory), CD-R, CD-R/W, a solid-state memory (e.g., a mask ROM, a PROM (Programmable ROM), an EPROM (Erasable PROM), a flash ROM, a RAM (Random Access Memory)). The program may also be provided to the computer by any type of a transitory computer readable medium. Examples of the transitory computer readable medium include an electrical signal, an optical signal, and an electromagnetic wave. The transitory computer readable medium can provide the program to the computer through a wired channel such as wires and optical fibers or a wireless channel.

The whole or a part of the example embodiments (including modifications, the same shall apply hereinafter) described above can be described as, but not limited to, the following Supplementary Notes.

### [Supplementary Note 1]

An information processing device comprising:
a stress value acquisition means configured to acquire a stress value representing a degree of stress of a target person;
a stress release action detection means configured to detect a stress release action, which is an action for releasing a stress, based on the stress value; and
a notification means configured to make a notification of a detection result of the stress release action.

### [Supplementary Note 2]

The information processing device according to Supplementary Note 1, further comprising
an exercise amount acquisition means configured to acquire an amount of exercise of the target person corresponding to the stress value,
wherein the stress release action detection means is configured to detect the stress release action based on the stress value and the amount of exercise.

### [Supplementary Note 3]

The information processing device according to Supplementary Note 1 or 2, further comprising
a life information acquisition means configured to acquire life information regarding a life of the target person,
wherein the stress release action detection means is configured to detect the stress release action based on the stress value and the life information.

### [Supplementary Note 4]

The information processing device according to Supplementary Note 3,
wherein the life information at least indicates a daily amount of exercise of the target person, and
wherein the stress release action detection means is configured to detect the stress release action based on
   a normalized amount of exercise that is obtained by normalizing the amount of exercise of the target person corresponding to the stress value by the daily amount of exercise and
   the stress value.

### [Supplementary Note 5]

The information processing device according to any one of Supplementary Notes 1 to 4,
wherein the stress release action detection means is configured to detect the stress release action based on
   an index having a positive correlation with the stress value and a negative correlation with the amount of exercise of the target person corresponding to the stress value, or
   an index having a negative correlation with the stress value and a positive correlation with the amount of exercise.

### [Supplementary Note 6]

The information processing device according to Supplementary Note 5,
wherein the stress release action detection means is configured to detect the stress release action based on
   the index and
   a lower limit threshold value and an upper limit threshold value for the index.

### [Supplementary Note 7]

The information processing device according to Supplementary Note 5 or 6,
wherein the stress release action detection means is configured to detect the stress release action based on
   the index and
   the stress value after a lapse of a predetermined time.

### [Supplementary Note 8]

The information processing device according to Supplementary Note 5,
wherein the stress release action detection means is configured to detect the stress release action based on a temporal change in the index.

### [Supplementary Note 9]

The information processing device according to any one of Supplementary Notes 5 to 8,
wherein the stress release action detection means is configured to detect the stress release action based on the stress value if it is determined, based on the index, that the target person is in a passive state in which the amount of exercise has increased due to a passive activity of the target person.

### [Supplementary Note 10]

The information processing device according to any one of Supplementary Notes 1 to 9, further comprising
an attribute information acquisition means configured to acquire attribute information relating to an attribute of the target person,
wherein the stress release action detection means is configured to detect the stress release action based on the stress value and the attribute information.

### [Supplementary Note 11]

The information processing device according to Supplementary Note 10,
wherein the attribute information at least includes information regarding whether or not the target person likes or dislikes exercises, and
wherein the stress release action detection means is configured to detect the stress release action based on
   the amount of exercise of the target person corresponding to the stress value,
   the information regarding whether or not the target person likes or dislikes exercises, and
   the stress value.

### [Supplementary Note 12]

The information processing device according to any one of Supplementary Notes 1 to 11,
wherein the stress release action detection means is configured to detect the stress release action which belongs to a recreation type, and
wherein the notification means is configured to make the notification regarding the detection result of the stress release action which belongs to the recreation type.

### [Supplementary Note 13]

A control method executed by a computer, the control method comprising:
acquiring a stress value representing a degree of stress of a target person;
detecting a stress release action, which is an action for releasing a stress, based on the stress value; and
making a notification of a detection result of the stress release action.

### [Supplementary Note 14]

A storage medium storing a program executed by a computer, the program causing the computer to
acquire a stress value representing a degree of stress of a target person;
detect a stress release action, which is an action for releasing a stress, based on the stress value; and
make a notification of a detection result of the stress release action.

While the invention has been particularly shown and described with reference to example embodiments thereof, the invention is not limited to these example embodiments. It will be understood by those of ordinary skilled in the art that various changes in form and details may be made therein without departing from the spirit and scope of the present invention as defined by the claims. In other words, it is needless to say that the present invention includes various modifications that could be made by a person skilled in the art according to the entire disclosure including the scope of the claims, and the technical philosophy. All Patent and Non-Patent Literatures mentioned in this specification are incorporated by reference in its entirety.

### DESCRIPTION OF REFERENCE NUMERALS

1, 1A, 1X Information processing device
2 Input device
3 Output device
4 Storage device
5 Sensor
8 Terminal device
100,100A Stress release detection system

What is claimed is:

1. A stress release action detection device comprising:
at least one memory configured to store instructions; and
at least one processor configured to execute the instructions to:
acquire a stress value representing a degree of stress of a target person;
acquire a biological signal from a sensor which measures a target person;
calculate an amount of exercise of the target person corresponding to the stress value, based on the biological signal and an exercise amount calculation model that is a machine learning model;
detect a stress release action, which is an action for releasing a stress, based on the stress value and the amount of exercise; and
make a notification of a detection result of the stress release action.

2. (canceled)

3. The stress release action detection device according to claim 1,
wherein the at least one processor is configured to further execute the instructions to acquire life information regarding a life of the target person,
wherein the at least one processor is configured to execute the instructions to detect the stress release action based on the stress value and the life information.

4. The stress release action detection device according to claim 3,
wherein the life information at least indicates a daily amount of exercise of the target person, and
wherein the at least one processor is configured to execute the instructions to detect the stress release action based on
a normalized amount of exercise that is obtained by normalizing the amount of exercise of the target person corresponding to the stress value by the daily amount of exercise and
the stress value.

5. The stress release action detection device according to claim 1,
wherein the at least one processor is configured to execute the instructions to detect the stress release action based on
an index having a positive correlation with the stress value and a negative correlation with the amount of exercise of the target person corresponding to the stress value, or
an index having a negative correlation with the stress value and a positive correlation with the amount of exercise.

6. The stress release action detection device according to claim 5,
wherein the at least one processor is configured to execute the instructions to detect the stress release action based on
the index and
a lower limit threshold value and an upper limit threshold value for the index.

7. The stress release action detection device according to claim 5,
wherein the at least one processor is configured to execute the instructions to detect the stress release action based on
the index and
the stress value after a lapse of a predetermined time.

8. The stress release action detection device according to claim 5,
wherein the at least one processor is configured to execute the instructions to detect the stress release action based on a temporal change in the index.

9. The stress release action detection device according to claim 5,
wherein the at least one processor is configured to execute the instructions to detect the stress release action based on the stress value if it is determined, based on the index, that the target person is in a passive state in which the amount of exercise has increased due to a passive activity of the target person.

10. The stress release action detection device according to claim 1,
wherein the at least one processor is configured to further execute the instructions to acquire attribute information relating to an attribute of the target person,
wherein the at least one processor is configured to execute the instructions to detect the stress release action based on the stress value and the attribute information.

11. The stress release action detection device according to claim 10,
wherein the attribute information at least includes information regarding whether or not the target person likes or dislikes exercises, and
wherein the at least one processor is configured to execute the instructions to detect the stress release action based on
the amount of exercise of the target person corresponding to the stress value,
the information regarding whether or not the target person likes or dislikes exercises, and
the stress value.

12. The stress release action detection device according to claim 1,
wherein the at least one processor is configured to execute the instructions to detect the stress release action which belongs to a recreation type, and
wherein the at least one processor is configured to execute the instructions to make the notification regarding the detection result of the stress release action which belongs to the recreation type.

13. A stress release action detection method executed by a computer, the control method comprising:
acquiring a stress value representing a degree of stress of a target person;
acquiring a biological signal from a sensor which measures a target person;
calculating an amount of exercise of the target person corresponding to the stress value, based on the biological signal and an exercise amount calculation model that is a machine learning model;
detecting a stress release action, which is an action for releasing a stress, based on the stress value and the amount of exercise; and
making a notification of a detection result of the stress release action.

14. A non-transitory computer readable storage medium storing a program executed by a computer, the program causing the computer to acquire a stress value representing a degree of stress of a target person;
acquire a biological signal from a sensor which measures a target person;
calculate an amount of exercise of the target person corresponding to the stress value, based on the biological signal and an exercise amount calculation model that is a machine learning model;
detect a stress release action, which is an action for releasing a stress, based on the stress value and the amount of exercise; and
make a notification of a detection result of the stress release action.
